(19) United States
(12) Patent Application Publication
(10) Pub. No.: US 2025/0368936 A1
(43) Pub. Date: Dec. 4, 2025

(54) DEVICE AND METHOD FOR CULTURING GEOBACTER THAT PRODUCES ULTRA-HIGH CONDUCTIVITY BIO-NANOWIRES

(71) Applicants: BEIJING FORESTRY UNIVERSITY, Beijing (CN); QINGLIN CHUANGNENG (SHANGHAI) TECHNOLOGY CO., LTD, Shanghai (CN)

(72) Inventors: Yan DANG, Beijing (CN); Haoyong LI, Beijing (CN); Haiyu XU, Beijing (CN); Hongbin WU, Beijing (CN)

(21) Appl. No.: 18/997,085

(22) PCT Filed: Sep. 14, 2023
(86) PCT No.: PCT/CN2023/118817

§ 371 Date: Jan. 19, 2025

(30) Foreign Application Priority Data
Feb. 21, 2023 (CN) 202310140663.2

(51) Int. Cl. C12M 1/06; C12M 1/00; C12M 1/107; C12M 1/34; C12N 1/20; C12R 1/01

(52) U.S. Cl. CPC C12M 27/02; C12M 23/02; C12M 23/36; C12M 41/12; C12M 41/34; C12M 41/44; C12M 47/02; C12N 1/20; C12R 2001/01

(57) ABSTRACT

This application discloses a device and a method for culturing Geobacter that produces ultra-high conductivity bio-nanowires. The device comprises a tank, at least two liquid color sensors, at least two dissolved oxygen sensors, a liquid level sensor, and a stirring paddle. The top of the tank is provided with an air valve; an upper part of the tank is provided with a feed inlet, a lower part is provided with a culture medium outlet, and the bottom of the tank is provided with a discharge outlet. This application can recycle ferrous citrate, add substrates multiple times, cultivate Geobacter that grow ultra-high conductivity bio-nanowires in batches, and achieve the amplification and cultivation of a large number of Geobacter metallireducens in the same volume.

### TECHNICAL FIELD

This disclosure belongs to the technical field of microbial culture, and relates to a device and a method for culturing Geobacter that produces ultra-high conductivity bio-nanowires.

### BACKGROUND

Geobacter metallireducens is a model microorganism that has attracted much attention in the fields of biology, biogeochemistry, and environmental science. It can be used for the bioremediation of pollutants such as soil, mines, and water bodies. It can also be applied in bioenergy conversion and the sustainable production of a new type of green “electronic product”, which are bio-nanowires.

Geobacter metallireducens grows microbial nanowires, which are conductive filaments with a diameter of nanometer scale grown on cellular membranes. The protein nanowires produced by microorganisms play a unique role in microbial life activities and biotechnology. They can be used for remote electron transfer and can be used as an environmentally friendly and sustainable “electronic material”. If microbial nanowires can be extracted on a large scale and introduced into electronic devices as conductive or semiconductive biomaterials, new opportunities will be opened up in the fields of bioelectronics, bioenergy, and medicine.

Geobacter metallireducens is the first isolated microorganism with nanowires. The conductivity of the nanowires produced by Geobacter metallireducens is 277S/cm, which is thousands of times higher than that of nanowires produced by other Geobacter species. Geobacter metallireducens is the preferred species for extracting bio-nanowires. Obtaining sufficient biomass is the prerequisite for efficient and large-scale extraction of bio-nanowires. The cost of batch culturing Geobacter metallireducens is very high, with a cost of approximately 162 CNY per million cells, resulting in a cost of up to 1200 CNY per gram for extracting bio-nanowires with ultra-high conductivity from Geobacter metallireducens. This high cost is primarily due to the need for highly pure ferric citrate (such as BioReagent grade ferric citrate produced by Sigma) as an electron acceptor for the rapid growth ofGeobacter metallireducens. However, the cost of ferric citrate is as high as 980 CNY per 250 grams, accounting for 99% of the cost of the medium, which is the main component of the cost of Geobacter metallireducens medium. This significantly limits the large-scale industrialized batch cultivation of Geobacter metallireducens and, consequently, the low-cost and large-scale extraction of nano-biological wires. Therefore, how to batch cultivate Geobacter metallireducens with ultra-high conductive microbial nanowires at a low cost, in order to efficiently obtain high-purity microbial nanowires, is an urgent problem to be solved for the engineering application of bio-nanowires.

Patent CN112778422A discloses a method for preparing type IV pilin protein similar to the bio-nanowires of Geobacter metallireducens. This method utilizes protein engineering. The GFP-tagged pilus protein monomer was expressed and purified in a prokaryotic expression system, and then induced to form pilus. This method evaluates the ability of different precipitants and precipitation conditions to promote the self-assembly of pilin protein through a method similar to protein crystallization condition screening. However, the structure of the pilin protein prepared by this assembly method is difficult to keep consistent with the structure of the naturally grown pilin proteins of microorganisms, and it is difficult to ensure that the prepared pilin protein can perform its unique physiological functions. Additionally, this method has problem such as high cost, and long production cycles, making it unsuitable for large-scale industrial production.

The preferred method to maintain the structure and function of the nanowires is to extract bio-nanowires from Geobacter metallireducens. Batch culturing of Geobacter metallireducens that produces bio-nanowires with ultra-high conductivity serves as the foundation for batch extraction of these bio-nanowires. Therefore, it is necessary to provide a low-cost device and a method for batch culturingGeobacter species that produce nanowires with ultra-high conductivity, which is of great significance in reducing the extraction cost of bio-nanowires from Geobacter metallireducens and promoting their industrial application.

### SUMMARY

The purpose of this application is to provide a device and a preparation method for culturing Geobacter that produces ultra-high conductivity bio-nanowires.

The present application proposes a device for culturing Geobacter that produces ultra-high conductivity bio-nanowires, comprising:
a tank, at least two liquid color sensors, at least two dissolved oxygen sensors, a liquid level sensor, and a stirring paddle;
wherein, the top of the tank is provided with an air valve driven by an air pump; an upper part of the tank is provided with a feed inlet, a lower part is provided with a medium outlet, and the bottom of the tank is provided with a discharge outlet;
wherein, one of in the at least two liquid color sensors is provided on the inner wall of the tank above the upper edge of the discharge outlet, and the others are provided on the inner wall of the tank;
wherein, at least two dissolved oxygen sensors are provided on the inner wall of the tank from top to bottom;
wherein, the liquid level sensor is provided on the inner wall of the tank below the lower edge of the feed inlet;
wherein, the stirring paddle is provided in the tank and driven by a motor.

In the aforementioned device for culturing Geobacter that produces ultra-high conductivity biological nanowires, the material used to make the tank is selected from glass, plastic, or metal.

Except the liquid color sensor installed on the upper edge of the discharge outlet, each of the liquid color sensors and the dissolved oxygen sensors are oppositely provided on the inner wall of the tank and located on the same cross-section of the inner wall of the tank.

In the aforementioned device for culturing Geobacter that produces ultra-high conductivity bio-nanowires, wherein the bottom of the tank is shaped as a cone, and the discharge outlet is provided at the tip of the cone;
a valve and a driving pump are respectively provided on the feed inlet, the medium outlet, and the discharge outlet;
the top of the tank is further provided with a pressure balance valve to balance air pressure when liquid is feeding and discharging, and a gas collecting bag connected with the pressure balancing valve to collect discharged air.

In this application, the specific functions of the air valve and the pressure balance valve are as follows.
(1) When the device for culturing Geobacter that grows ultra-high conductivity bio-nanowires is used for the first time, nitrogen is introduced through the air valve to replace the air inside the device.

During the initial startup of the device for culturing Geobacter that grows ultra-high conductivity bio-nanowires, the air valve and the air pump are activated. At the same time, the pressure balance valve and the gas collection bag are also activated. Nitrogen is infused into the device to replace the existing air (if the air in the device is not replaced, it may increase the dissolved oxygen in the anaerobic medium, affecting the growth of Geobacter). During subsequent operations, when the device is already in an anaerobic environment, only the pressure balance valve needs to be activated.
(2) Infusing air through the air valve during iron cycling

During iron cycling, the air valve is opened, and air is infused into the device through the air pump. The oxygen in the air is utilized to oxidize ferrous iron. At the same time, the air valve and the air pump work together to control the amount of air infused into the device, ensuring that the oxygen in the infused air is consumed by the ferrous iron and does not cause an increase in the dissolved oxygen in the medium.

In the aforementioned device for culturing Geobacter that produces ultra-high conductivity bio-nanowires, wherein the liquid color sensors, the dissolved oxygen sensors, and the liquid level sensors are all connected to a main control computer for real-time monitoring and recording;
the air valve, the air pump, the valves, the driving pumps, and the pressure balance valve are all connected to the main control computer for centralized control;
the air valve, the pressure balance valve, the valves, and the pressure balancing valve are all provided with a 0.22-micron filter for filtering microorganisms in the air.

In the aforementioned device for culturing Geobacter that produces ultra-high conductivity bio-nanowires, wherein the device further comprises a medium reservoir, an acetate sodium reserve pool, a medium regeneration tank, a centrifuge, and a bio-nanowire extraction device; the bio-nanowire extraction device is configured to extract bio-nanowires.

The feed inlet is connected to the medium reservoir and the acetate sodium reserve pool; the medium outlet is connected to the medium regeneration tank; the discharge outlet expels the Geobacter cells, which are then collected by the centrifuge and sent to the bio-nanowire extraction device.

In the aforementioned device for culturing Geobacter that produces ultra-high conductivity bio-nanowires, wherein the number of liquid color sensors is 2 to 6, specifically it is can be 3; when the number of liquid color sensors is 2, one is provided on the inner wall of the tank above the upper edge of the feed outlet, and the other is provided on the inner wall of the tank; when the number of liquid color sensors is 3 to 6, one is provided on the inner wall of the tank above the upper edge of the feed outlet, and the others are provided on the inner wall of the tank from top to bottom in sequence;
the number of dissolved oxygen sensors is 2 to 6, specifically it is can be 3.

In the aforementioned device for culturing Geobacter that produces ultra-high conductivity bio-nanowires, wherein the stirring paddle extends into the bottom of the tank and connected to a differential gear to achieve operation at different rotational speeds.

In this disclosure, all the components of the device for culturing Geobacter that produces ultra-high conductivity bio-nanowires are either commonly known components in the field or existing components capable of achieving their respective functions.

The present application further provides a method for recycling ferric citrate to batch cultivation of Geobacter that produces ultra-high conductivity bio-nanowires using the device, comprising the following steps:
1) adding ferric citrate medium into the tank, so that the lowermost dissolved oxygen sensors and one of the liquid color sensors are located at half of the liquid level height, wherein, the dissolved oxygen sensors at half of the liquid level height detect a dissolved oxygen content of less than 0.2 mg/L, and the liquid color sensor at half of the liquid level height indicates a reddish-brown color at this time;
2) inoculating Geobacter metallireducens into the ferric citrate medium, activating the stirring paddle to mix them evenly, and then processing with conduct constant temperature cultivation at 30° C.;
wherein, during the constant temperature cultivation process, the ferric iron in the ferric citrate is reduced to ferrous iron, and the liquid color sensors provided on the inner wall of the tank initially indicate a black color and then a yellow color;
3) precipitating the Geobacter metallireducens and opening the air valve and the air pump to oxidize the ferrous iron after the liquid color sensor provided on the upper edge of the discharge outlet indicates a yellow color; when the liquid at half of the liquid level height is oxidized and the liquid color sensor provided on the corresponding level indicates a black color, and a dissolved oxygen content detected by the dissolved oxygen sensor does not affect the cultivation of the Geobacter metallireducens, deactivating the air pump, adding the prepared sodium acetate stock solution and letting it stand;
4) repeating steps 2 to 3, without the step of inoculating the Geobacter metallireducens, oxidizing the ferrous iron in the ferric citrate medium by the air to regenerate the ferric citrate, and expanding the same volume of ferric citrate medium to cultivate the Geobacter metallireducens, naturally precipitating the Geobacter metallireducens bacterial cell, discharging them from the discharge outlet, and collecting the bacterial cell into the bio-nanowire extraction device for nanowire extraction by the centrifuge.

In this application, the height of the liquid level detected by the liquid level sensor is used to control the volume of the ferrous citrate medium being added. The addition of the ferrous citrate medium is stopped when liquid is detected by the liquid level sensor.

In the aforementioned method, the initial inoculation amount of the Geobacter metallireducens is 10% to 20% in step 2;

In the step 3 of the aforementioned method, when the liquid is oxidized at half of the liquid level height and the liquid color sensor indicates a black color, a dissolved oxygen level, detected by the dissolved oxygen sensor provided on the inner wall of the tank from top to bottom, indicates 0 to 0.5 mg/L; specifically, 0 to 0.3 mg/L

In the aforementioned method, after step 3, the dissolved oxygen sensor indicates that the dissolved oxygen level has reached 0.0 mg/L.

In the aforementioned method, when the number of dissolved oxygen sensors is 3, the dissolved oxygen sensors provided on the inner wall of the tank from top to bottom, respectively indicate dissolved oxygen levels of DO<0.3 mg/L, DO<0.2 mg/L, and DO=0.0+0.05 mg/L.

In this application, the metal-reducing Geobacter refers to the Geobacter metallireducens species, specifically the product named “Geobacter metallireducens” commercially available from the German DSMZ company (website: www.dsmz.de) with the product catalog number DSM 7210.

In this application, in step 1, the liquid color sensor indicates a reddish-brown color at this time, with specific RGB values of R=114±10, G=66±10, B=40±10;
in step 2, the liquid color sensor indicates a black color, with specific RGB values of R=39±10, G=29±10, B=23±10; the liquid color sensor indicates a yellow color, with specific RGB values of R=201±20, G=161±10, B=69±10;
in step 3, when liquid at half of the liquid level height is oxidized, the liquid color sensor indicates a black color, with specific RGB values of R=39±10, G=29±10, B=23±10.

The specific mechanisms involved in the cultivation process from step 2 to 3 in this application are shown as follows. During the constant temperature cultivation at 30° C., as the Geobacter metallireducens grows, the ferric iron in the sole electron acceptor ferric citrate is reduced to ferrous iron; the intermediate state of the transformation from ferric citrate to ferrous iron causes the liquid color sensor to indicate a black color (e.g., RGB: R=39±10, G=29±10, B=23±10); as the growth continues, the ferric citrate is further reduced to ferrous iron, and under the combined effect of all substances' colors in the medium, the liquid color sensor indicates a yellow color (e.g., RGB: R=201±20, G=161±10, B=69±10). At this point, the microbial cells are sedimented, and the air pump is turned on. Under the oxidative effect of air, and the joint control of the dissolved oxygen sensor, color sensor, air valve and air pump, the ferrous iron is gradually oxidized. When liquid at half of the liquid level height is oxidized, the liquid color sensor indicates a black color (e.g., RGB: R=39±10, G=29±10, B=23±10). At this moment, the dissolved oxygen sensors installed on the inner wall of the tank from top to bottom should indicate dissolved oxygen levels of DO<0.3 mg/L, DO<0.2 mg/L, and DO=0.0+0.05 mg/L, respectively. The air pump is then turned off, and a prepared sodium acetate stock solution is added, followed by resting. The dissolved oxygen at this point oxidizes the ferrous iron in the medium back to ferric iron. Controlling the dissolved oxygen within the aforementioned ranges ensures that it does not affect the cultivation of anaerobic Geobacter metallireducens, and further utilization of the dissolved oxygen under the action of the remaining ferrous iron in the medium, ultimately reducing DO to approximately 0.0 mg/L.

In the aforementioned method, the number of repetitions in step 4 can range from 3 to 20 times, specifically 10 times. Multiple repetitions facilitate the cultivation of a larger biomass, which reduces the subsequent cost of extracting pilin. The more repetitions, the lower the cost becomes.

In this application, the stirring paddle is connected to the different ialgear and stirs at a rotational speed ranging from 5 to 20 r/min for 1 to 2 minutes. The purpose of this stirring is to uniformly distribute the oxidized ferric iron and bacterial cells throughout the device, enabling faster growth of Geobacter metallireducens. However, the stirring speed should not be excessive, as vigorous stirring may rupture the bacterial cells, which is detrimental to bacterial growth.

In the aforementioned method, wherein the ferric citrate medium is a liquid medium comprising the following concentration components:
ferric citrate of 2.74±0.5 g/L; sodium nitrate of 0.425±0.04 g/L; sodium acetate of 6.8±2 g/L;
NaH₂PO₄ at 0.58 g/L, NH₄Cl at 0.25 g/L, KCl at 0.10 g/L, NaHCO₃ at 2.50 g/L, 1 mmol/L of Na₂SeO₄ solution at 1 mL/L;
trace element solution of 10 mL/L;
10 mL/L of vitamin solution;
wherein, the trace element solution consists of the following concentration components: 1.5 g/L of nitrilotriacetic acid, 1.46 g/L of MgSO₄, 1.0 g/L of NaCl, 0.13 g/L of ZnCl₂, 0.45 g/L of MnSO₄, 0.05 g/L of FeSO₄, 0.08 g/L of CaCl₂, 0.022 g/L of CoCl₂, 0.01 g/L of H₃BO₃, 0.0064 g/L of CuSO₄, 0.0054 g/L of Kal (SO₄)₂, 0.023 g/L of Na₂WO₄, 0.021 g/L of Na₂MoO₄, and 0.020 g/L of NiCl₂;
the vitamin solution consists of the following concentration components: 0.002 g/L of biotin, 0.005 g/L of pantothenic acid, 0.0001 g/L of vitamin B12, 0.005 g/L of p-aminobenzoic acid, 0.005 g/L of lipoic acid, 0.005 g/L of niacin, 0.005 g/L of thiamine, 0.005 g/L of vitamin B2, 0.01 g/L of vitamin B6, and 0.002 g/L of folic acid.

The present application further provides a ferric citrate medium for culturing Geobacter that produces ultra-high conductivity bio-nanowires, wherein the ferric citrate medium is a liquid medium comprising the following concentration components:
2.74±0.5 g/L of ferric citrate; 0.425±0.04 g/L of sodium nitrate; 6.8±2 g/L of sodium acetate;
0.58 g/L of NaH₂PO₄, 0.25 g/L of NH₄Cl, 0.10 g/L of KCl, 2.50 g/L of NaHCO₃; 1 mL/L of 1 mmol/L Na₂SeO₄ solution;
10 mL/L of trace element solution;
10 mL/L of vitamin solution;
wherein, the trace element solution consists of following concentration components: 1.5 g/L of nitrilotriacetic acid, 1.46 g/L of MgSO₄, 1.0 g/L of NaCl, 0.13 g/L of ZnCl₂, 0.45 g/L of MnSO₄, 0.05 g/L of FeSO₄, 0.08 g/L of CaCl₂, 0.022 g/L of CoCl₂, 0.01 g/L of H₃BO₃, 0.0064 g/L of CuSO₄, 0.0054 g/L of Kal (SO₄)₂, 0.023 g/L of Na₂WO₄, 0.021 g/L of Na₂MoO₄, and 0.020 g/L of NiCl₂;
the vitamin solution consists of the following concentration components: 0.002 g/L of biotin, 0.005 g/L of pantothenic acid, 0.0001 g/L of vitamin B12, 0.005 g/L of p-aminobenzoic acid, 0.005 g/L of lipoic acid, 0.005 g/L of niacin, 0.005 g/L of thiamine, 0.005 g/L of vitamin B2, 0.01 g/L of vitamin B6, and 0.002g/L of folic acid.

### BRIEF DESCRIPTION OF THE DRAWINGS

FIG. 1 is a structural diagram of the device for culturing Geobacter with ultra-high conductivity bio-nanowires in the embodiment of the present application.
Reference Numbers in FIG. 1:
1. Air pump; 2. Air valve; 3. Feed inlet; 4. Feed valve; 5. Feed water pump; 6-1, 6-2, 6-3, 6-4 Liquid color sensors; 7. Medium outlet; 8. Water pump for medium Drainage; 9. Discharge valve; 10. Bacterial cell discharge pump; 11. Discharge valve; 12. Stirring paddle; 13. Motor; 14. Differential gear; 15. Pressure balance valve; 16. Liquid level sensor; 17-1, 17-2, 17-3 Dissolved oxygen sensors; 18. Discharge outlet; 23. Gas collection bag.

FIG. 2 presents a comparison of the growth status of Geobacter bacterial cell cultured using the present application's method for the cyclic utilization of ferric citrate for batch cultivation of Geobacter bacterial cell with ultra-high conductivity bio-nanowires, versus the traditional cultivation method. Specifically, FIG. 2a depicts the growth status of Geobacter bacterial cell cultured using the traditional method, while FIG. 2b shows the growth status of Geobacter bacterial cell cultured using the application's method of the present disclosure.

FIG. 3 presents the cryo-electron microscopy image of the ultra-high conductivity bio-nanowires extracted from Geobacter metallireducens in this application.

FIG. 4 illustrates the relationship between the number of cycles of ferric citrate utilization, and biomass and the unit cell cultivation cost in this application. Specifically, FIG. 4a depicts the correlation between the number of cycles and cell quantity, while FIG. 4b shows the relationship between the number of cycles and the unit cost of cell cultivation.

FIG. 5 illustrates the mechanism diagram of the present application for recycling cultivating Geobacter bacterial cell to regenerate ferric citrate.

FIG. 6 presents the results of the relative expression levels of bio-nanowire monomer genes in the present application's production of bio-nanowires, compared to those of the control and existing methods.

FIG. 7 shows the electron micrograph of bio-nanowires extracted from bacterial cell cultured in an existing citric acid iron medium.

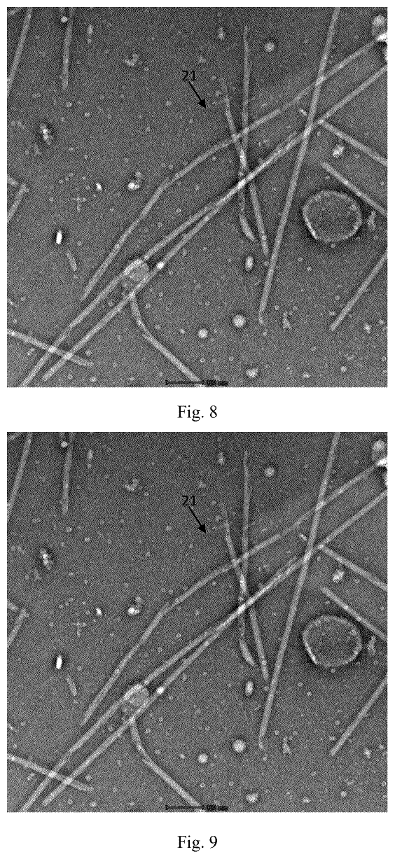

FIG. 8 shows the electron micrograph of bio-nanowires extracted from bacterial cell cultured in the optimized citric acid iron medium of the present application.
Reference Numbers in FIGS. 7-8:
21. Bio-nanowire, 22. Impurity.

### DETAILED DESCRIPTION

Unless otherwise specified, the experimental methods used in the following embodiments are conventional methods.

The materials, reagents, etc. used in the following embodiments can be obtained from commercial sources unless otherwise specified.

The reaction principles adopted in this application include:

1. Geobacter metallireducens reduces ferric iron

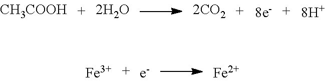

2. Air oxidizes ferrous iron to ferric iron, which continues to serve as an electron acceptor.

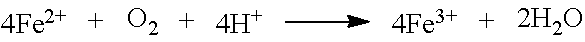

The embodiment of the present disclosure provides a low-cost method for batch culturing Geobacter with ultra-high conductivity bio-nanowires, which utilizes controlled air oxidation to convert the metabolic product ferrous iron of Geobacter metallireducens into ferric iron. The ferric iron can then continue to serve as the essential electron acceptor for the growth of Geobacter metallireducens, enabling further expansion of the culture. This method achieves multiple recycling of ferric iron at an extremely low cost with one-time input, thereby significantly reducing the costs associated with the amplification culture of Geobacter metallireducens and the extraction of ultra-conductive microbial nanowires.

The example provided by the present disclosure for low-cost batch culturing of Geobacter metallireducens with ultra-high conductivity bio-nanowires, is described in detail and in conjunction with FIG. 1.

### EXAMPLE 1

As shown in FIG. 1, this embodiment provides a bulk cultivation device for Geobacter metallireducens with a specific working volume of 1 liter. It comprises an air pump 1, an air valve 2, a feed water pump 5, a water pump for medium Drainage 8, a bacterial cell discharge pump 10, a stirring paddle 12, a motor 13, a differential gear 14, a pressure balance valve 15, liquid color sensors 6-1, 6-2, 6-3, 6-4, a liquid level sensor 16, dissolved oxygen sensors 17-1, 17-2, 17-3, and a gas collection bag 23.

For the first-time operation, the discharge outlet 18, the discharge valve 9, the feed valve 4, and the air valve 2 are closed. The pressure balance valve 15, the air pump 1 and the air valve 2 are switched on to fill the device with nitrogen, allowing air to be expelled through the pressure balance valve 15, creating an anaerobic environment inside the device.

The feed valve 4 and the feed water pump 5 are switched on to introduce the prepared anaerobic medium into the device, which uses ferric citrate as the sole electron acceptor and sodium acetate as the sole electron donor. When the liquid level sensor 16 detects the liquid, the feed valve 4 and the feed water pump 5 are switched off.

At this moment, the dissolved oxygen sensors 17-1, 17-2, and 17-3 should indicate a dissolved oxygen level of less than 0.2 mg/L, and all color sensors 6-1, 6-2, 6-3, and 6-4 should indicate a reddish-brown color (RGB: R=114, G=66, B=40).

The feed valve 4 is switched on, and the feed water pump 5 is used to inoculate approximately 100 ml of Geobacter metallireducens (specific inoculation percentage is 10%) into the device. Then, the feed valve 4 and the feed water pump 5 are switched off.

The motor 13 is switched on and the differential gear 14 is adjusted to make the stirring paddle 12 rotate at a speed of 10 r/min for 5 minutes, so that the inoculated bacterial cell are evenly distributed in the medium.

The device is maintained at a constant temperature of 30° C., and the motor 13 is turned on once per 12 hours to adjust the differential gear 14, causing the stirring paddle 12 to rotate at a speed of 10 r/min for 5 minutes.

As the Geobacter metallireducens grows, the ferric citrate acting as the sole electron acceptor, is gradually reduced, and the liquid color sensors 6-1, 6-2, 6-3 indicate a black color (RGB: R=39, G=29, B=23). As the Geobacter metallireducens continues to grow, the ferric iron in the ferric citrate is reduced to ferrous iron, the liquid color sensors 6-1, 6-2, 6-3 subsequently indicate a yellow color (RGB: R=201, G=161, B=69) in conjunction with other components in the medium. At this point, the electron acceptor for the ferric citrate, has been consumed almost entirely, and the Geobacter metallireducens cannot continue to grow.

The stirring paddle 12 is switched off and the liquid is rested for 12 hours to allow the bacterial cells to be precipitated to the bottom of the device.

The valve 2, the air pump 1, and the pressure balance valve 15 are switched on to slowly pump air into the device. As the air comes into contact with the medium, the ferrous ions are gradually oxidized. At this point, the liquid color sensors 6-1, 6-2, and 6-3 will sequentially indicate colors from yellow (RGB: R=201, G=161, B=69) to black (RGB: R=39, G=29, B=23). Additionally, the readings of the dissolved oxygen sensors 17-1, 17-2, and 17-3 will also sequentially increase. When the dissolved oxygen sensor 17-1 indicates DO<0.3 mg/L, 17-2 indicates DO<0.2 mg/L, and 17-3 indicates DO=0.0 mg/L, valve 2 and deactivate air pump 1 are switched off.

The feed valve 4 and the feed water pump 5 are switched on to add the prepared anaerobic sodium acetate solution, and the pressure-balancing valve 15 is switched off.

The liquid is rested for 1 hour until the ferrous ions in the device fully consume the oxygen in the headspace. Then the motor 13 is switched on, the differential gear 14 is adjusted, and the stirring paddle 12 is set to stir at a speed of 10 r/min for 2 minutes.

The aforementioned process of culture is repeated at a constant temperature until the liquid color sensors 6-1, 6-2, and 6-3 indicates a yellow color (RGB: R=201, G=161, B=69). At this point, the electron acceptor for the ferric iron has been consumed once again, and the Geobacter metallireducens cannot continue to grow.

The aforementioned steps of bacterial cell sedimentation, pumping in air, adding anaerobic sodium acetate solution, and culture at a constant temperature are repeated for 3 to 20 times (specifically, 10 times for example). Through this process, the same amount of medium can be used to amplify and cultivate several times more bacterial cells than before, significantly reducing the cost of amplification and cultivation.

When the liquid color sensors 6-1, 6-2, and 6-3 indicate yellow (RGB: R=201, G=161, B=69), the liquid is stopped to be stirred and the bacterial cells are allowed to be naturally precipitated for 24 hours.

When the liquid color sensor 6-4 indicates red (RGB: R=150, G=80, B=60), the discharge valve 11, the pressure balance valve 15 and the bacterial cell discharge pump 10 are switched on to discharge the bacterial cell precipitated at the bottom of the conical collection tank. 30 ml (approximately half of the conical tank's capacity) of the bacterial cell is discharged. The remaining Geobacter metallireducens can be used as microorganisms for continued operation and inoculation. The discharged bacterial solution is centrifuged at 8000 g for 1 minute using a centrifuge to collect the bacterial cell. The collected bacterial cell enters the biological nanowire extraction device, while the supernatant centrifuged is collected and enters the medium regeneration tank for reuse after regeneration.

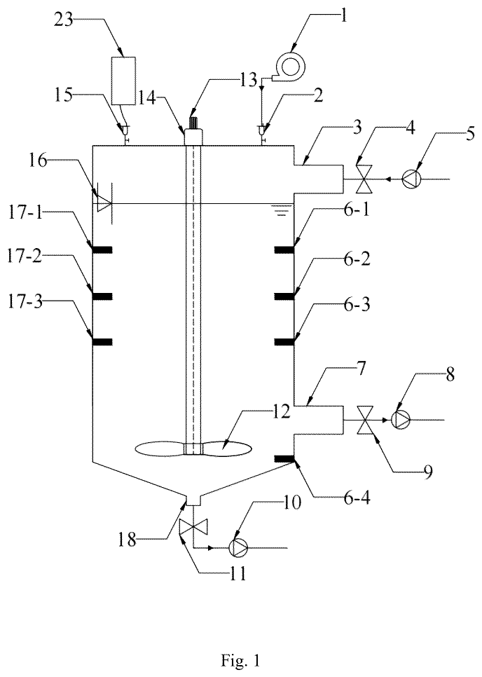

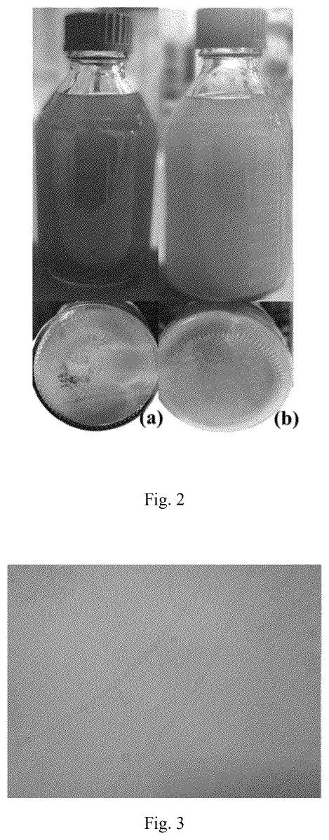

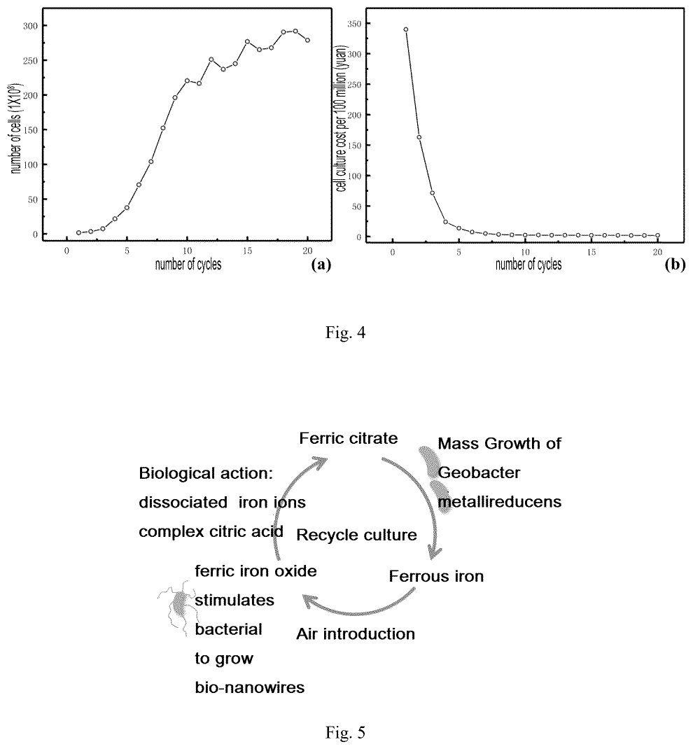

This method is executed for 10 times, the cultured Geobacter metallireducens can be enriched significantly. As shown in FIG. 2, FIG. 2a represents the growth of Geobacter metallireducens using traditional cultivation methods, with approximately 1.66×10⁸ Geobacter metallireducens cells per milliliter of bacterial solution, and the cost of culturing every 100 million cells is approximately 340 yuan. As depicted in FIG. 2b, the growth of Geobacter metallireducens using the present method is shown, with a significant enrichment of red Geobacter metallireducens, reaching approximately 2.78×10¹⁰ cells per milliliter of bacterial solution; as shown in FIG. 4b, the cost per 100 million cells is reduced to approximately 2 yuan.

Currently, there are no reported cases of achieving high-purity Geobacter metallireducens nanowires at low cost. Some literature has reported methods for extracting nanowires from Geobacter sulfurreducens, with a cost of approximately 53,000 yuan per gram of nanowires. However, the present disclosure enables the acquisition of a large quantity of Geobacter metallireducens at low cost. After two cycles, 0.046 grams of ultra-high conductivity nanowires can be extracted from every liter of bacterial solution, significantly enhancing the extraction efficiency of these nanowires. Employing the ferric citrate cycling method outlined in this disclosure drastically reduces the cost of extracting bio-nanowires. The aforementioned method is executed for 20 times, the cost of nanowires can be reduced to approximately 12 yuan per gram. Furthermore, as evidenced by the cryo-electron microscopy images in FIG. 3, the extracted nanowires exhibit excellent purity.

As depicted in FIG. 5, the regeneration process of ferric citrate in this disclosure is not just a simple process of ferrous iron oxidizing to ferric iron. The traditional preparation process of ferric citrate usually requires high temperatures and the control of various reactant ratios. In contrast, the cyclic regeneration of ferric citrate outlined in this disclosure involves the participation ofGeobacter species, wherein the iron within the oxidized ferric iron oxide is dissociated under mild conditions (neutral pH and approximately 30° C.), and then it complexes with citric acid again to form ferric citrate, meaning that Geobacter also participates in the regeneration process of ferric citrate. Moreover, the regeneration and utilization of ferric citrate constitute an adaptive dynamic equilibrium process mediated and influenced by biological entities. Apart from the control method described in this disclosure, there is no requirement for real-time precise regulation of the proportions of reactants such as iron and citric acid to achieve the regeneration of ferric citrate, thus achieving the effect of cultivating abundant large amount of Geobacter metallireducens with ultra-high conductivity bio-nanowires. A regeneration rate of ferric citrate per cycle is no less than 60% in this embodiment.

### EXAMPLE 2

The medium composition is shown as follows:
2.74 g/L of ferric citrate; 0.425 g/L of sodium nitrate; 6.8 g/L of sodium acetate;

The addition of a certain amount of sodium nitrate not only provides an additional electron donor but also generates ammonia nitrogen as a metabolite, which serves as a nitrogen source for microbial growth, thus eliminating the need for extra ammonia nitrogen supplementation.
0.58 g/L of NaH₂PO₄; 0.25 g/L of NH₄Cl; 0.10 g/L of KCl; 2.50 g/L of NaHCO₃; 1 mL/L of 1 mmol/L Na₂SeO₄ solution;
10 mL/L of trace element solution (composition of trace element solution: 1.5 g/L of aminotriacetic acid, 1.46 g/L of MgSO₄, 1.0 g/L of NaCl, 0.13 g/L of ZnCl₂, 0.45 g/L of MnSO₄, 0.05 g/L of FeSO₄, 0.08 g/L of CaCl₂, 0.022 g/L of CoCl₂, 0.01 g/L of H₃BO₃, 0.0064 g/L of CuSO₄, 0.0054 g/L of Kal (SO₄)₂, 0.023 g/L of Na₂WO₄, 0.021 g/L of Na₂MoO₄, 0.020 g/L of NiCl₂);
10 mL/L of vitamin solution (composition of vitamin solution: 0.002 g/L of biotin, 0.005 g/L of pantothenic acid, 0.0001 g/L of vitamin B12, 0.005 g/L of p-aminobenzoic acid, 0.005 g/L of lipoic acid, 0.005 g/L of niacin, 0.005 g/L of thiamine, 0.005 g/L of vitamin B2, 0.01 g/L of vitamin B6, 0.002 g/L of folic acid).

### Medium Preparation Method:

1. First, solution A is prepared, its composition is shown as below:
NaH₂PO₄: 0.58 g,NH₄Cl: 0.25 g,KCl: 0.10 g, NaHCO₃: 2.50 g, 1 mL of 1 mmol/L Na₂SeO₄ solution; 10 mL of trace element solution; 10 mL of Vitamin solution.

The solution is diluted to 1000 ml.

2. The preparation process of ferric citrate medium is as follows in detail: Firstly, 13.7g of ferric citrate is weighted for later use. A 1-liter beaker is filled with 150 mL of deionized water and placed on a heating stirrer. Once the water boils, a small amount of ferric citrate is added, and after the added ferric citrate is completely dissolved, the remaining ferric citrate is added in a small amount and more times, while maintaining a state of stirring and heating as the reagent is added. Once all the ferric citrate has been dissolved, the solution is transferred to another beaker containing 600 mL of deionized water and allowed to cool to room temperature. At this point, the pH value of the solution will be around 1.8. The pH value is adjusted to 6.0-6.5 using 10 mol/L NaOH (when the pH value approaches 5.0, lower concentration of 5 mol/L or 2 mol/L NaOH is used for finer adjustments). After the solution has cooled down, the following components are added sequentially: 0.58g of NaH₂PO₄, 0.25g of NH₄Cl, 0.10g of KCl, 2.50g of NaHCO₃, 1 mL of 1 mmol/L Na₂SeO₄ solution, 10mL of trace element solution (with the same composition as mentioned), and 10 mL of vitamin solution (with the same composition as well). Finally, the solution is diluted to 1000 ml.

3. Solution A and the ferric citrate medium are mixed uniformly in a volume ratio of 4:1.

4. It is aerated for 30 minutes, producing a mixed gas of carbon dioxide and nitrogen (CO₂/N₂= 20/80).

5. After deoxygenation, the mixture is sterilized at 121° C. under high pressure for 30 minutes, then it is cooled to room temperature for later use.

The pH values of the two solutions are adjusted separately before being mixed, the stability of the final pH value of the prepared medium is ensured.

Bio-nanowires of Geobacter metallireducens are obtained through the cultivation method described in Embodiment 1 of this application by using the above-mentioned medium.

In Embodiment 1 of the present disclosure, the concentration of ferric citrate in the traditional culture medium used is 13.7 g/L. Further optimization of the culture medium composition was conducted in Embodiment 2 of this disclosure, where the concentration of ferric citrate is reduced to 2.74 g/L. As depicted in the comparison of electron micrographs of the biological nanowires extracted from the bacterial cells grown in the existing culture medium and the optimized ferric citrate culture medium of Embodiment 2 in FIGS. 7-8, it can be seen that the optimized culture medium composition in Embodiment 2 of the present disclosure, by reducing the content of ferric citrate, not only decreases costs but also minimizes contamination during the extraction of biological nanowires. Since the culture medium can be recycled in this disclosure, the reduction in ferric citrate content does not affect the number of bacterial cells cultured, but rather, compared to the control, it increases the number of bacterial cells, achieving the same cultivation effect as Embodiment 1.

The specific experimental method for measuring the gene expression level of biological nanowires is shown as follows:

The single-use ferric citrate medium group serves as the comparative method for this disclosure.

The insoluble amorphous ferric iron group, i.e., the existing method (following the method described in US2006257985A1) is shown as below:
a medium containing the following components is prepared (units: grams per liter of deionized water): NaHCO₃, 2.5; CaCl₂·2H₂O, 0.1; KCl, 0.1; NH₄Cl, 1.5; NaH₂PO₄, 0.58; CH₃COONa, 6.8. Vitamins and trace minerals are added from a stock solution (the above-mentioned components are the same as in this disclosure). Approximately 200 mmol/L of amorphous ferric iron oxide is added to the medium, which is then sterilized under high pressure. The medium is inoculated with 10% Geobacter metallireducens and incubated at a constant temperature of 37° C.

The recycled ferric citrate medium group of this disclosure follows the method described in Embodiment 1 of the disclosure.

RNA extraction is performed using the RNeasy PowerSoil Total RNA kit from QIAGEN, followed by purification of the RNA samples with the DNA-free DNase (Ambion) kit. The purified RNA was then reverse-transcribed into cDNA using the TRUEscript 1st Strand cDNA Synthesis Kit from Aidlab Biotechnologies Co., Ltd. qRT-PCR testing was conducted using SYBR Green I fluorescent dye, with proC as the internal reference gene.

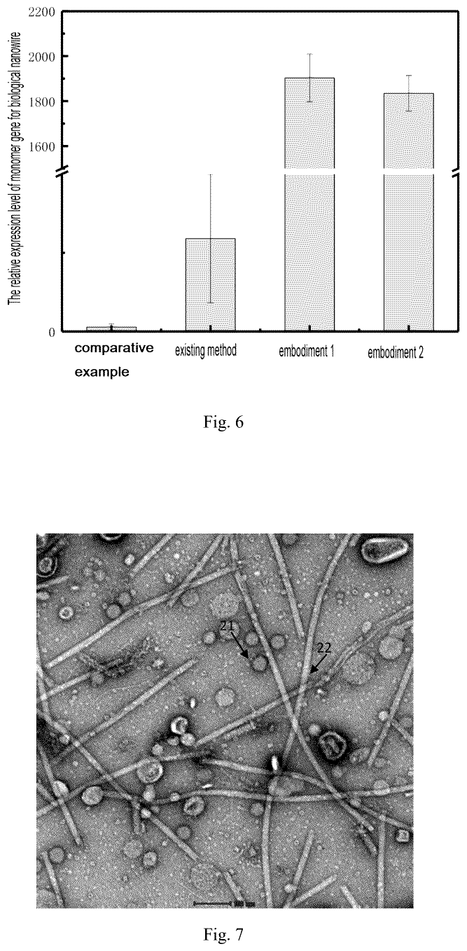

The results of the gene expression level measurement for biological nanowires are shown in FIG. 6. As evident from the results in FIG. 6, the method of the present disclosure compared to the control group using ferric citrate alone for cultivation increased the production of bio-nanowires by 730%. The relative expression level of the gene encoding the monomer of bio-nanowires was more than 400 times higher compared to using ferric citrate alone, and nearly 20 times higher compared to the existing method (following the method described in US2006257985A1) using insoluble amorphous ferric iron.

Comparative Example The setup and method in this control group are identical to those in Embodiment 1 of the present disclosure, with the exception that this comparative example adopts a traditional culturing method. Specifically, the air pump 1 and the air valve 2 are closed, and the step of slowly pumping air into the device is omitted. As a result, there is no contact between air and the medium. During the culturing process, it was observed that the liquid color sensors 6-1, 6-2, 6-3 remained yellow and did not turn black, indicating that the process of ferrous iron being gradually oxidized did not occur. Furthermore, the readings of the dissolved oxygen sensors 17-1, 17-2, 17-3 remained unchanged, confirming that there was no recycling of ferric citrate during the culturing process. The results are shown in FIG. 2a, where the growth of Geobacter metallireducens was approximately 1.66×10⁸ cells per milliliter of bacterial solution. The cost per one hundred million cells cultured is approximately 340 yuan, as depicted in FIG. 4b.

The aforementioned experimental results demonstrate that the present disclosure employs a ferric citrate medium to cultivate Geobacter metallireducens, utilizing trace amounts of air to oxidize the metabolite Fe (II) of the costly electron acceptor ferric citrate. This enables ferric citrate to be recycled after a single addition into the device, thereby achieving low-cost, large-scale, and efficient cultivation of Geobacter metallireducens that produces bio-nanowires with ultra-high conductivity.

### Industrial Applications

1. The present disclosure utilizes controlled air oxidation of ferrous iron to regenerate ferric iron, which can then complex with citric acid to reform ferric citrate as an electron acceptor for the growth of Geobacter metallireducens. By controlling the speed and extent of the air oxidation of ferrous iron, the bacterial cells are first precipitated to the bottom of the device, and the ferrous iron is gradually oxidized by air into insoluble amorphous ferric iron oxide. The oxidation process is halted when half of the liquid height is oxidized (at this point, the color sensor at the ½ liquid level indicates black with RGB values of R=39±10, G=29±10, B=23±10). This approach not only oxidizes ferrous iron but also consumes the introduced oxygen in the device, regenerating ferric iron. As a result, there is no risk of bacterial cell death due to increased dissolved oxygen levels. Furthermore, the bacterial cells, stimulated by the insoluble amorphous ferric iron oxide, produce a large amount of biological nanowires. During the growth process, the insoluble amorphous ferric iron oxide slowly ionizes and complexes with citric acid to form ferric citrate, achieving the regeneration of ferric citrate (in this disclosure, Geobacter metallireducens participates in the regeneration of ferric citrate). Thus, the present disclosure enables the cost-effective reuse of the expensive electron acceptor ferric citrate at minimal expense.

2. By repeatedly adding substrates, the amplification and cultivation of a large number of Geobacter metallireducens in the same volume of medium is achieved, significantly increasing the concentration of ultra-high conductivity nanowires. This allows for the subsequent extraction step to obtain more ultra-high conductivity nanowires, reducing costs while enhancing the extraction efficiency of these nanowires.

3. Current research has found that Geobacter metallireducens grows and reproduces at the fastest rate in liquid culture media using ferric citrate as an electron acceptor. However, in prior art, the bacterial cells cultured in ferric citrate medium barely produce bio-nanowires, and this biological characteristic of Geobacter metallireducens fundamentally limits the mass production of bio-nanowires. The present disclosure ingeniously addresses these issues from a fundamental perspective, overcoming the technical bottleneck that prevents the industrial production of bio-nanowires from Geobacter metallireducens. In this disclosure, upon initial operation, after Geobacter metallireducens are inoculated in the device, the Geobacter metallireducens rapidly proliferate using ferric citrate as an electron acceptor, yielding a large amount of bacterial cells. During the growth process of bacterial cells in the present invention, the insoluble amorphous ferric iron oxide slowly ionizes and recombines with citric acid to regenerate ferric citrate. During this regeneration process, the bacterial cells are stimulated by the insoluble amorphous ferric iron oxide to produce a large number of bio-nanowires. Not only does this cycling of the medium enable low-cost, large-scale, and environmentally friendly cultivation of Geobacter metallireducens, but the cycling process also allows the bacterial cell to utilize ferric citrate for prolific cell growth, while simultaneously stimulating the production of abundant bio-nanowires.

Geobacter metallireducens participates in both the regeneration of ferric citrate and the cycling process of the medium. Furthermore, utilizing soluble ferric citrate for the cultivation of the bacterial cell does not contaminate the subsequent extraction process of bio-nanowires. Consequently, this approach not only enables the acquisition of a large quantity of Geobacter metallireducens cells but also facilitates the growth of bio-nanowires by these cells.

1. A device for culturing Geobacter that produces ultra-high conductivity bio-nanowires, comprising:
a tank, at least two liquid color sensors, at least two dissolved oxygen sensors, a liquid level sensor, and a stirring paddle;
wherein, the top of the tank is provided with an air valve driven by an air pump; an upper part of the tank is provided with a feed inlet, a lower part is provided with a medium outlet, and the bottom of the tank is provided with a discharge outlet;
wherein, one of in the at least two liquid color sensors is provided on the inner wall of the tank above the upper edge of the discharge outlet, and the others are provided on the inner wall of the tank;
wherein, at least two dissolved oxygen sensors are provided on the inner wall of the tank from top to bottom;
wherein, the liquid level sensor is provided on the inner wall of the tank below the lower edge of the feed inlet;
wherein, the stirring paddle is provided in the tank and driven by a motor.

2. The device for culturing Geobacter that produces ultra-high conductivity bio-nanowires according to claim 1, wherein
a material for making the tank is selected from glass, plastic or metal; one liquid color sensor is provided on the upper edge of the discharge port, each of other liquid color sensors and each of the dissolved oxygen sensors are provided opposite to each other on the inner wall of the tank, and are located on the same cross-section of the inner wall of the tank.

3. The device for culturing Geobacter that produces ultra-high conductivity bio-nanowires according to claim 2, wherein
the bottom of the tank is shaped as a cone, and the discharge outlet is provided at the tip of the cone;
a valve and a driving pump are respectively provided on the feed inlet, the medium outlet, and the discharge outlet; the top of the tank is further provided with a pressure balance valve and a gas collecting bag connected with the pressure balancing valve.

4. The device for culturingGeobacter that produces ultra-high conductivity bio-nanowires according to claim 3, wherein
the liquid color sensors, the dissolved oxygen sensors, and the liquid level sensors are all connected to a main control computer for real-time monitoring and recording;
the air valve, the air pump, the valves, the driving pumps, and the pressure balance valve are all connected to the main control computer for centralized control;
the air valve, the pressure balance valve, the valves, and the pressure balance valve are all provided with a 0.22-micron filter for filtering microorganisms in the air.

5. The device for culturing Geobacter that produces ultra-high conductivity bio-nanowires according to claim 4, wherein
the device further comprises a medium reservoir, an acetate sodium reserve pool, a medium regeneration tank, a centrifuge, and a bio-nanowire extraction device; the bio- nanowire extraction device is configured to extract bio-nanowires;
the feed inlet is connected to the medium reservoir and the acetate sodium reserve pool; the medium outlet is connected to the medium regeneration tank; the discharge outlet expels the Geobacter cells, which are then collected by the centrifuge and sent to the bio-nanowire extraction device.

6. The device for culturing Geobacter that produces ultra-high conductivity bio-nanowires according to claim 1, wherein
the number of liquid color sensors is 2 to 6; when the number of liquid color sensors is 2, one is provided on the inner wall of the tank above the upper edge of the feed outlet, and the other is provided on the inner wall of the tank; when the number of liquid color sensors is 3 to 6, one is provided on the inner wall of the tank above the upper edge of the feed outlet, and the others are provided on the inner wall of the tank from top to bottom in sequence;
the number of dissolved oxygen sensors is 2 to 6;
the stirring paddle extends into the bottom of the tank and connected to a differential gear to achieve operation at different rotational speeds.

7. A method for recycling ferric citrate to batch cultivation of Geobacter that produces ultra-high conductivity bio-nanowires using the device according to claim 1 comprising the following steps:
1.) adding ferric citrate medium into the tank to make the dissolved oxygen sensors and one of the liquid color sensors at half of the liquid level height, wherein, the dissolved oxygen sensors at half of the liquid level height detects a dissolved oxygen content of less than 0.2 mg/L, and the liquid color sensor at half of the liquid level height indicates a reddish-brown color at this time;
2.) inoculating Geobacter metallireducens into the ferric citrate medium, activating the stirring paddle to mix them evenly, and then processing with conduct constant temperature cultivation at 30° C.;
wherein, during the constant temperature cultivation process, the ferric iron in the ferric citrate is reduced to ferrous iron, and the liquid color sensors provided on the inner wall of the tank initially indicate a black color and then a yellow color;
3.) precipitating the Geobacter metallireducens and turning on the air valve and the air pump to oxidize the ferrous iron after the liquid color sensor provided on the upper edge of the discharge outlet indicates a yellow color; when the liquid at half of the liquid level height is oxidized and the liquid color sensor provided on the corresponding level indicates a black color, and a dissolved oxygen content detected by the dissolved oxygen sensor does not affect the cultivation of the Geobacter metallireducens, turning off the air pump, adding the prepared sodium acetate stock solution, and resting;
repeating steps 2 to 3, without the step of inoculating the Geobacter metallireducens, oxidizing the ferrous iron in the ferric citrate medium by the air to regenerate the ferric citrate, and expanding the same volume of ferric citrate medium to cultivate the Geobacter metallireducens, naturally precipitating the Geobacter metallireducens, discharging them from the discharge outlet, and collecting the cells into the bio-nanowire extraction device for nanowire extraction by the centrifuge.

8. The method according to claim 7, wherein the initial inoculation amount of the Geobacter metallireducens is 10% to 20% in step 2;
in step 3, when the liquid is oxidized at half of the liquid level height and the liquid color sensor indicates a black color, a dissolved oxygen level, detected by the dissolved oxygen sensor provided on the inner wall of the tank from top to bottom, indicates 0 to 0.5 mg/L;
after step 3 is completed, the dissolved oxygen sensor indicates that the dissolved oxygen level has reached 0.0 mg/L.

9. The method according to claim 7, wherein
when the number of dissolved oxygen sensors is 3, and the liquid is oxidized at half of the liquid level height and the liquid color sensor indicates a black color, the dissolved oxygen sensors provided on the inner wall of the tank from top to bottom, respectively indicate dissolved oxygen levels of DO<0.3 mg/L, DO<0.2 mg/L, and DO=0.0+0.05 mg/L.

10. The method according to claim 7, wherein the step 4 is repeated for 3 to 20 times.

11-12. (canceled)
